(19) United States
(12) Patent Application Publication
(10) Pub. No.: US 2006/0008536 A1
(43) Pub. Date: Jan. 12, 2006

(54) Pharmaceutical compositions and methods of use

(75) Inventors: William Richard Cates, Ackerly, TX (US); L. Randall Denton, Dallas, TX (US)

Correspondence Address: Jeffrey L. Wendt, 34 Driftoak Circle, The Woodlands, TX, 77381, US

(73) Assignee: JDC Pharmaceutical, Dallas, TX (US)

(21) Appl. No.: 10/887,537

(22) Filed: Jul. 8, 2004

(51) Int. Cl. A61K 33/32

(52) U.S. Cl. 424/641

(57) ABSTRACT

Compositions, kits including ingredients of the compositions, and methods useful in preventing and treating skin, mucosal tissue, and anorectal discomfort are presented, the compositions comprising an aromatic alcohol, an inorganic vasoconstrictor, an emulsifying agent, and a composition comprising one or more esters of fatty acids.

### BACKGROUND INFORMATION

1. Technical Field

The present invention relates in general to pharmaceutical compositions and methods of use, and more particularly to pharmaceutical compositions and methods of use in preventing or treating skin conditions such as sunburn, skin abrasions, acne, and hemorrhoids and other anorectic conditions and symptoms.

2. Background Art

Hemorrhoids (varicosities of the veins of the hemorrhoidal plexus, often complicated by inflammation, thrombosis, and bleeding), and anal fissures an acute longitudinal tear or a chronic ovoid ulcer in the stratified squamous epithelium of the anal canal) are problematic in a significant amount of the human population, and hemorrhoids are almost universally present in children and adults, according to the Merck Manual of Diagnosis and Therapy, Section 3 “Gastrointestinal Disorders”, Chapter 35, “Anorectal Disorders”, online at http://www.merck.com/mrkshared/mmanual/section3/chapter 35/35b.isp, accessed Mar. 22, 2004. As noted in this manual, hemorrhoids (also known as piles) are often asymptomatic but may cause bleeding, protrusion, and pain. Recommended treatment includes stool softeners or bulking agents (e.g., psyllium) to correct constipation and straining, thus allowing hemorrhoids to resolve. The manual also notes that “pain caused by a thrombosed hemorrhoid can be treated with reassurance, warm sitz baths, anesthetic ointments, or witch hazel (hamamelis) compresses.” If bleeding hemorrhoids are present, it is suggested to treat them by injection sclerotherapy with 5% phenol in vegetable oil. Larger internal hemorrhoids or those that fail to respond to injection sclerotherapy are suggested to be treated by rubber band ligation. Most of these treatments are well known to physicians; unfortunately, “reassurance” may not be adequate for many patients suffering pain.

Pfenninger, John L., and Zainea, George G., M.D., Common Anorectal Conditions: Part II. Lesions”, Am Fam Physician 2001 ; 64:77-88, discuss treatment of hemorrhoids, and refers to a published comprehensive review of the evaluation and treatment of hemorrhoids, Pjenninger JL, Surrell J., Nonsurgical treatment options for internal hemorrhoids, Am Fam Physician 1995; 52:821-34,839-41. In the former reference, the authors note that thrombosed hemorrhoids are typically treated by incision under local anesthetic. to remove the clot, and conservative therapy with sitz baths and pain medication may be indicated, although recovery is prolonged. The authors note that there is a higher recurrence rate when simple incision and expression of the clot is practiced, and whichever surgical technique is used, the entire thrombus must be removed, as hemorrhoids contain septate channels and, if the entire clot is not removed, thrombosis will reaccumulate. around the nucleus of retained clot.

The problems presented by hemorrhoids and other anorectal conditions have prompted many remedies. A perusal of the international patent literature leads to many interesting compositions, none of which disclose or suggest the compositions of the present invention. U.S. Pat. No. 439,581 discloses an ointment comprising sweet oil of almonds (24 oz.), white wax (4 oz.), spermaceti (4 oz.), sub-nitrate of bismuth (2 oz.), glycerin (2 oz.), and carbolic acid crystals (7 drams). Tannic acid is added for bleeding hemorrhoids in the proportion of 1 part, by weight, of tannic acid to 10 parts by weight of the ointment. In these weight proportions, without the addition of tannic acid, the ointment has 2.66 weight percent carbolic acid (phenol) and 73 weight percent sweet oil of almonds. With the addition of tannic acid, the proportions are 2.42 weight percent carbolic acid and 66 weight percent sweet oil of almonds. The patent provides no guidance as to the functions of the various ingredients, and there is no suggestion of the use of lanolin.

Other United States patents include U.S. Pat. Nos. 1,676,554; 1,674,353; 4,613,498; 4,169,143; 4,514,384; 4,518,583; 5,627,216; 6,228,387; 4,118,480; 5,858,371; 5,196,405; 6,322,820; and 6,699,488. Internationally, mention can be made of the following patents: Russian patent 2179451; French patent publication no, 2177626; Romanian patent no. 114552 B; Romanian patent no. 116522 B; and Russian patent 178047.

Of all of the above references, only the 439,581 patent discloses an ointment comprising sweet oil of almonds and carbolic acid (phenol), but there is no mention of lanolin. Other references, such as U.S. Pat. No. 4,118,480, disclose a preparation for treating hemorrhoids and anal fissures comprising phenol in an ointment base of lanolin or petrolatum, however there is no mention of a natural oil emulsifier such as sweet oil of almonds. The compositions of Russian patent 178047 include zinc oxide and lanolin, but do not discuss using a natural oil emulsifier such as sweet oil of almond. Accordingly, the need remains for a composition that can provide improved relief for pain, swelling, and other symptoms, while exhibiting pleasing tactile properties and decreased allergic activity. It is desired to provide compositions and methods of use which prevent and/or relieve the pain and discomfort associated with skin and mucosal tissue discomforts, such as sunburn, acne, hemorrhoids, piles, anal fissures, and similar anorectal conditions which utilize the beneficial qualities of lanolin-like materials and a natural oil emulsifier, such as sweet almond oil. It is a further desire to provide a composition that includes vasoconstrictive and anesthetic properties, that is relatively simple to apply to the area to be treated, and that may include adjuvants, such as perftimes, thickeners, and other pharmacologically acceptable ingredients. It has now been unexpectedly found that compositions comprising a high percentage of lanolin-like ingredients show excellent therapeutic and preventative benefits, with low or no allergic activity, and are stable.

### SUMMARY OF THE INVENTION

In accordance with the present invention, pharmaceutical compositions and therapeutic methods of use are presented which reduce or overcome many of the problems known in the art. As used herein, “composition” includes but is not limited to creams, lotions, balms, foams, salves, gels, solids, ointments, liquid and other formulations. When a composition of the invention is packaged in combination with an applicator, such as a pad, bandage, gauze, compress, and the like, an inventive kit is formed. Therefore, the inventive compositions are useful for treating a variety of conditions under a variety of circumstances, in addition to anorectal conditions, and these uses are deemed to be within the literal scope of the invention as defined by the claims herein.

A first aspect of the invention is a composition having protective or therapeutic function comprising:
a) an aromatic alcohol or derivative;
b) an inorganic vasoconstrictor;
c) an emulsifying agent; and
d) a composition comprising an ester of one or more fatty acids.

A preferred aromatic alcohol is phenol, while a preferred emulsifying agent is naturally derived sweet almond oil. Zinc oxide is a preferred inorganic vasoconstrictor. Lanolin is one preferred composition comprising an ester of one or more fatty acids, lanolin itself comprising cholesterol esters of higher fatty acids, and is available in hydrogenated, ethoxylated and acetylated versions.

A second aspect of the invention comprises preventative and therapeutic methods of using the inventive compositions of the first aspect of the invention to prevent or treat skin or mucosal tissue discomfort, for example from sunburn, windburn, chemical burns, scrapes, abrasions and floor burns (as might happen in indoor and outdoor sporting events, such as basketball games, soccer matches, and the like), acne, and especially to treat anorectal conditions, such as hemorrhoids, the inventive methods comprising the steps of formulating a composition in accordance with the first aspect of the invention and applying the composition to the distressed area of the skin, mucosal or anorectal region. One application may be sufficient, but the invention covers situations where repetitive application may be necessary, depending on the particular composition, severity of the discomfort being treated, and patient tolerance of the composition. Preferred methods are those wherein the formulation step comprises mixing the aromatic alcohol and inorganic vasoconstrictor ingredients into the composition comprising a cholesterol ester of fatty acid, and then adding the emulsifying agent and mixing to emulsify the composition to form a cream or salve.

A third aspect of the invention is a kit comprising a composition of the first aspect of the invention with an applicator. Kits of the invention are useful as components of safety kits carried by consumer and medical vehicles, for example.

Further aspects and advantages of the invention will become apparent by reviewing the description of embodiments that follows.

### DESCRIPTION OF PREFERRED EMBODIMENTS

As previously mentioned, there have been a variety of remedies for hemorrhoids over the years, and probably have been for centuries. Many doctors prescribe surgery for hemorrhoids and other anorectal diseases; however, with the ever rising cost of medical treatment and surgery today, and with the expected rise in cost of these procedures and shifting of cost to the consumer through reduced coverage and/or more expensive insurance premiums, the inventive compositions have been devised as either a prescription pharmaceutical or over the counter pharmaceutical that can easily be applied by the patient or the patient's guardian, without need of expensive surgery.

Compositions of the invention, in their broadest sense, comprise an aromatic alcohol, an inorganic vasoconstrictor, an emulsifying agent, and a composition comprising one or more cholesterol esters of higher fatty acids.

The aromatic alcohol finctions primarily as an antiseptic and disinfectant, and secondarily as a local anesthetic. The terms “aromatic alcohol”, “phenol” and “hydroxybenzene” are used interchangeably herein as describing a class of organic compounds and their derivatives in which one or more hydroxy groups (—OH) are attached directly to an aromatic ring structure. They have the generic formula R(OH)ₙ, where R is selected from the group consisting of aryl, aryl alkyl, aryl halide, dialkylaminoaryl, aryl acids, aryl aldehyde, naphthyl and the like, and combinations and mixtures thereof, and n ranges from I up to the maximum number of hydroxyl groups able to be substituted on the molecule. More preferably n ranges from 1 to 3. The aromatic alcohol may also be used in its esterified form, and with mild application of heat and in the presence of moisture converted into the aromatic alcohol form. Similarly, an allyl phenyl ether could be used, and through application of mild heat, a Claisen rearrangement mechanism would provide either the o-allyl phenol or p-allyl phenol.

Examples of suitable aromatic alcohols useful in the invention are phenol (otherwise known as hydroxybenzene, benzophenol, carbolic acid, and phenylic acid), the cresols, the xylenols (otherwise known as dimethylphenol, hydroxydimethylbenzene, and dimethylhydroxybenzene), compounds where n=2 such as resorcinol, catechol, and hydroquinone, compounds where n=3 such as pyrogallol (1,2,3-trihydroxybenzene) and phloroglucinol, and naphthols such as α-naphthol (1-naphthol) and β-naphthol (2-naphthol), and combinations and mixtures thereof. Useful derivatives might include resorcinol acetate and resorcinol monobenzoate, and salt forms of various aromatic acids. While many phenols and derivatives may be used, care must be taken in choosing the R group to avoid compounds such as urushiols, which are active constituents of the allergic oil of poison ivy and probably also of poison oak.

The aromatic alcohol is present in the inventive compositions in an amount ranging from about 1 weight percent up to about 10 weight percent, either as a single compound or combination of two or more compounds. While it is not necessarily known if this ingredient reacts with the other components, reactivity is allowed, as long as the essential antiseptic function of the aromatic alcohol is maintained, even if only for a brief time. If a derivative is used, such as an allyl phenyl ether, as mentioned herein mild application of heat would produce the aromatic alcohol. The amount may have to be adjusted up or down, depending on the individual, as many phenols are skin irritants when exposed in concentrated form.

Vasoconstrictors are known in the pharmaceutical art and their function and structure requires little explanation to the skilled artisan. The function is to narrow the blood vessels so that less blood is able to flow through at a time. An inorganic vasoconstrictor is preferred in the present invention, although some organic constituent (in the molecule, or admixed therewith) is allowed, as long as the vasoconstrictive function is maintained, and allergic reaction is not experienced. In particular, zinc compounds are particularly preferred, especially zinc oxide (ZnO). Other zinc compounds that may be employed include zinc acetate, zinc bacitracin, zinc carbonate, zinc chloride, zinc iodide, zinc chloroiodides (mixtures of zinc chloride and zinc iodide), zinc hydroxide, zinc oxalate, zinc permanganate, zinc peroxide, and combinations and mixtures thereof. The inorganic vasoconstrictor is present in an amount ranging from about 5 weight percent up to about 15 weight percent, more preferably from about 5 weight percent to about 8 weight percent. Although some references employ zinc oxide as a protectant, this is not the primary function in the inventive compositions; however, at the higher weight percentages the zinc compound will have some degree of protection from UV. This is obviously more important in therapeutic (or preventative) uses not related to anorectal symptoms, such as scrapes from sporting events, preventing or healing sunburn, and the like. It is not expected that the vasoconstrictor will react with any of the other ingredients, but reaction is acceptable as long as the essential vasoconstrictive function is maintained for a sufficient time period. The measurement of a sufficient time period depends on the therapeutic or preventive use, the patient, and the particular inventive composition employed, and may depend on the shelf life of a particular composition if made previous to use. (One advantage of the kits of the invention is that the inventive composition may be made on site.) In general, the goal is to balance having to apply the composition too often to be a nuisance, and curing or alleviating the symptoms.

An emulsifying agent is used to emulsify the compositions of the invention, essentially to form a cream, paste, lotion, salve, foam, gel, or similar form of composition. Natural oil emusifiers are preferred, especially those containing substantial amounts of diglycerides and/or triglycerides. Non-limiting examples of natural emulsifiers comprising diglycerides and/or triglycerides suitable for use herein include almond oil, castor oil, soy bean oil, derivatized soybean oils such as maleated soy bean oil, safflower oil, cotton seed oil, corn oil, walnut oil, peanut oil, olive oil, cod liver oil, avocado oil, palm oil and sesame oil, vegetable oils, sunflower seed oil, and vegetable oil derivatives; coconut oil and derivatized coconut oil, cottonseed oil and derivatized cottonseed oil, jojoba oil, cocoa butter, and combinations thereof. In addition any of the above oils that have been partially or fully hydrogenated are also suitable. Sweet almond oil is preferred. The amount of emulsifying agent used depends on the consistency and type of composition desired. The emulsifying agent is preferably present in compositions of the invention in an amount ranging from about 10 weight percent to about 30 weight percent, more particularly from about 20 to about 30 weight percent if a higher degree of emulsification is desired.

The composition comprising one or more esters of fatty acids may comprise any esters of one or more alcohols and one or more fatty acids that provide the function of a vehicle for the compositions of the invention, and are preferably not derived from petroleum. Fatty acids useful herein are those having from about 10 to about 30 carbon atoms, preferably from about 12 to about 22 carbon atoms, and more preferably from about 16 to about 22 carbon atoms. These fatty acids are preferably fully saturated and can be straight or branched chain acids. Also included are diacids, triacids, and other multiple acids which meet the requirements herein. Also included herein are salts of these fatty acids. Nonlimiting examples of fatty acids include lauric acid, palmitic acid, stearic acid, behenic acid, sebacic acid, and mixtures thereof. The alcohol portion of the esters may be any alcohol having from about 10 to 50 carbon atoms, more preferably from about 10 to about 30 carbon atoms, including fully saturated and mildly unsaturated alcohols, and can be straight or branched chain alcohols. Nonlimiting examples of fatty alcohols include, cetyl alcohol, stearyl alcohol, behenyl alcohol, and mixtures thereof. Also useful are the sterols (steroid alcohols) having the common steroid nucleus, plus an 8-10 carbon atom side chain and a hydroxyl group. Lanolin, a known emollient used in face creams, facial tissues, and the like, is a composition derived from solvent treatment of wool, comprising cholesterol esters of higher fatty acids, and is available in hydrogenated, ethoxylated and acetylated derivatives, as well as in hydrated and anhydrous forms. One reason for preferring use of lanolin and lanolin-derivatives is to avoid petroleum oils and waxes, due to their actual or perceived allergic properties, cost, or a combination of these reasons. The anhydrous form can be mixed with about twice its weight of water without separation. Non-limiting examples of lanolin and lanolin derivatives suitable for use herein include lanolin, lanolin oil, lanolin wax, lanolin alcohols, lanolin fatty acids, isopropyl lanolate, acetylated lanolin, acetylated lanolin alcohols, lanolin alcohol linoleate, lanolin alcohol riconoleate, hydroxylated lanolin, hydrogenated lanolin and combinations thereof. The composition comprising an ester of one or more fatty acids is preferably present at a weight percentage ranging from about 50 to about 70 weight percent.

In preparing the desired pharmaceutical form of the compositions of the present inventions, various diluents, additives, and adjuvants may be utilized. For example, the inventive compositions may comprise perfumes, surfactants, ointment type bases, propellants, thickening agents, humectants, silicone-type fluids and solid diluents as is known in the art.

The inventive compositions may be applied either by hand, an applicator or other means, depending on the particular patient and form of the composition. It is recommended to wash the irritated area prior to applying the product. The product may be applied any time of the day or night. The preparation of the inventive composition may be carried out according to conventional methods for storing the composition for future use. Alternatively, the compositions of the invention may be made just prior to use, by using kits of the invention. That is, the four primary ingredients may be packaged separately and mixed when needed, or two or three ingredients pre-mixed, combining the remaining ingredients on-site just prior to application. Kits of the invention may be preferred in certain compositions to enhance shelf life of the compositions. The four primary ingredients and any auxiliary or carrier substances may be suitably mixed by stirring or homogenization using conventional devices. The incorporation of the ingredients can be carried out at elevated temperatures in order to facilitate the process. Temperatures ranging from room temperature (about 25° C.) up to about and 90° C. may be employed during mixing of the ingredients, with cooling back down to room temperature or lower for the final preparation. As some of the components may change color upon exposure to air and/or sunlight, it is recommended that the compositions of the invention be stored in dark cabinets, although this is not strictly necessary.

From the foregoing detailed description of specific embodiments of the invention, it should be apparent that novel and patentable compositions, kits, and methods of using same have been described. Although specific embodiments of the invention have been described herein in some detail, this has been done solely for the purposes of describing various features and aspects of the invention, and is not intended to be limiting with respect to the scope of the invention. It is contemplated that various substitutions, alterations, and/or modifications, including but not limited to those implementation variations which may have been suggested herein, may be made to the described embodiments without departing from the scope of the appended claims. For example, various optional ingredients may be utilized, and variations in the viscosity, odor, color, consistency, packaging, and the like are considered within the invention.

What is claimed is:

1. A composition comprising:
a) an aromatic alcohol;
b) an inorganic vasoconstrictor;
c) an emulsifying agent; and
d) a composition comprising one or more esters of fatty acids.

2. The composition in accordance with claim I wherein the aromatic alcohol is selected from the group consisting of compounds within the generic formula R(OH)ₙ, where R is selected from the group consisting of aryl, aryl alkyl, aryl halide, dialkylaminoaryl, aryl acids, aryl aldehyde, naphthyl and the like, and combinations and mixtures thereof, and n ranges from 1 up to a maximum number of hydroxyl groups able to be substituted on the compound.

3. The composition in accordance with claim 2 wherein the aromatic alcohol is phenol.

4. The composition in accordance with claim 1 wherein the inorganic vasoconstrictor is selected from the group consisting of zinc oxide, zinc acetate, zinc bacitracin, zinc carbonate, zinc chloride, zinc iodide, zinc chloroiodides, zinc hydroxide, zinc oxalate, zinc permanganate, zinc peroxide, and combinations and mixtures thereof.

5. The composition in accordance with claim 4 wherein the inorganic vasoconstrictor is zinc oxide.

6. The composition in accordance with claim 1 wherein the emulsifying agent is a natural oil selected from the group consisting of almond oil, castor oil, soy bean oil, derivatized soybean oil, safflower oil, cotton seed oil, corn oil, walnut oil, peanut oil, olive oil, cod liver oil, avocado oil, palm oil, sesame oil, vegetable oils, sunflower seed oil, vegetable oil derivatives, coconut oil, derivatized coconut oil, cottonseed oil, derivatized cottonseed oil, jojoba oil, cocoa butter, and combinations and mixture thereof.

7. The composition in accordance with claim 6 wherein the natural oil is sweet almond oil.

8. The composition in accordance with claim 1 wherein the aromatic alcohol is present in an amount ranging from about 1 to about 10 weight percent.

9. The composition in accordance with claim 1 wherein the inorganic vasoconstrictor is present in an amount ranging from about 5 to about 15 weight percent.

10. The composition in accordance with claim 1 wherein the emulsifying agent is present in an amount ranging from about 10 to about 30 weight percent.

11. The composition in accordance with claim 1 wherein the composition comprising esters of fatty acids is present in an amount ranging from about 50 to about 70 weight percent.

12. A composition for treating anorectal discomfort, the composition comprising:
a) from about 1 to about 10 weight percent of an aromatic alcohol;
b) from about 5 to about 15 weight percent of an inorganic vasoconstrictor;
c) from about 10 to about 30 weight percent of an emulsifying agent; and
d) from about 50 to about 70 weight percent of lanolin.

13. A composition comprising:
a) from about 1 to about 10 weight percent phenol;
b) from about 5 to about 15 weight percent of zinc oxide;
c) from about 10 to about 30 weight percent of sweet almond oil; and
d) from about 50 to about 70 weight percent of lanolin.

14. A kit comprising the composition of claim 1 and an applicator.

15. A kit comprising the composition of claim 12 and an applicator.

16. A kit comprising the composition of claim 13 and an applicator.

17. A kit comprising a first container having an aromatic alcohol therein, a second container having an inorganic vasoconstrictor therein, a third container having an emulsifier therein, and a fourth container having a composition comprising an ester of a fatty acid therein.

18. A kit comprising a first container having therein an aromatic alcohol combined with a composition comprising an ester of a fatty acid, a second container having an inorganic vasoconstrictor therein, and a third container having an emulsifier therein.

19. A method of preventing skin or mucosal tissue discomfort, the method comprising the steps of formulating the composition of claim 1 and applying the composition to an area of skin or mucosal tissue.

20. A method of treating skin or mucosal tissue discomfort, the method comprising the steps of formulating the composition of claim 1 and applying the composition to a distressed area of skin or mucosal tissue.

21. A method of treating anorectal discomfort, the method comprising the steps of formulating the composition of claim 1 and applying the composition to a distressed area of the anorectal region.

22. A method of treating anorectal discomfort, the method comprising the steps of formulating the composition of claim 12 and applying the composition to a distressed area of the anorectal region.

23. A method of treating anorectal discomfort, the method comprising the steps of formulating the composition of claim 13 and applying the composition to a distressed area of the anorectal region.
